(19) United States
(12) Patent Application Publication
(10) Pub. No.: US 2024/0428410 A1
(43) Pub. Date: Dec. 26, 2024

(54) MEDICAL ASSISTANCE SYSTEM AND IMAGE DISPLAY METHOD

(71) Applicant: OLYMPUS MEDICAL SYSTEMS CORP., Tokyo (JP)

(72) Inventors: Takashi NAGATA, Tokyo (JP); Shiho MIYAUCHI, Tokyo (JP); Kazuya WATANABE, Tokyo (JP); Ryo OGUMA, Tokyo (JP); Satomi KOBAYASHI, Tokyo (JP); Kazuya FURUHO, Tokyo (JP); Isao TATESHITA, Tokyo (JP)

(73) Assignee: OLYMPUS MEDICAL SYSTEMS CORP., Tokyo (JP)

(21) Appl. No.: 18/823,012

(22) Filed: Sep. 3, 2024

Related U.S. Application Data
(63) Continuation of application No. PCT/JP2022/009059, filed on Mar. 3, 2022.

(51) Int. Cl. G06T 7/00; G06F 3/14; G16H 30/20

(52) U.S. Cl. CPC G06T 7/0012; G06F 3/14; G16H 30/20; G06T 2207/10068; G06T 2207/30096

(57) ABSTRACT

An image group specifying unit specifies one or more image groups including a plurality of lesion image showing a lesion from among a plurality of images acquired during examination. A display control unit displays the plurality of images included in the image groups at a first display frame rate and displays a plurality of images not included in the image groups at a second display frame rate higher than the first display frame rate. The display control unit may display only some of the plurality of images not included in the image groups and hide the rest of the images.

### CROSS-REFERENCE TO RELATED APPLICATIONS

This application is based upon and claims the benefit of priority from the International Application No. PCT/JP2022/009059, filed on Mar. 3, 2022, the entire contents of which are incorporated.

### BACKGROUND

### 1. Technical Field

The present disclosure relates to a medical assistance system and an image display method for displaying images acquired during examination.

### 2. Description of the Related Art

In endoscopic examination, a doctor observes endoscopic images displayed on a display device and, when an image containing a lesion is displayed or an image containing a predetermined observation target such as the entrance of a site of an organ is displayed, operates an endoscope release switch to capture (save) the endoscopic image. After the examination is completed, the doctor observes (interprets) captured images again. Thus, if a large number of images are captured, the time and effort required for observing the images increase.

JP 2006-280792 discloses an image display device that displays a series of images captured in a time series. The image display device disclosed in JP2006-280792 detects, from a series of images, a continuous image group of continuous images having correlative values of a plurality of pixel regions between adjacent images that are equal to or greater than a predetermined value, specifies one or more representative images from the continuous image group, and displays the remaining images other than the representative images at a display frame rate faster than that of the representative images.

### SUMMARY

A medical assistance system according to one embodiment of the present disclosure includes: a processor comprising hardware. The processor is configured to: specify one or more image groups including at least one lesion image showing a lesion from among a plurality of images acquired during examination, the image groups being configured in time series starting from an image in which the lesion is first framed; display the image groups at a first display frame rate; and display an image different from those in the image groups among the plurality of images at a second display frame rate higher than the first display frame rate or display the plurality of images different from those in the image groups in a thinned-out manner.

Another embodiment of the present disclosure relates to an image display method including: specifying one or more image groups including at least one lesion image showing a lesion from among a plurality of images acquired during examination, the image groups being configured in time series starting from an image in which the lesion is first framed; displaying the image groups at a first display frame rate; and displaying an image different from those in the image groups among the plurality of images at a second display frame rate higher than the first display frame rate or display the plurality of images different from those in the image groups in a thinned-out manner.

Optional combinations of the aforementioned constituting elements and implementations of the present disclosure in the form of methods, apparatuses, systems, recording mediums, and computer programs may also be practiced as additional modes of the present disclosure.

### BRIEF DESCRIPTION OF THE DRAWINGS

Embodiments will now be described, by way of example only, with reference to the accompanying drawings which are meant to be exemplary, not limiting, and wherein like elements are numbered alike in several Figures, in which:

FIG. 1 is a diagram showing the configuration of a medical assistance system according to an embodiment;

FIG. 2 is a diagram showing functional blocks of a server device;

FIG. 3 is a diagram showing functional blocks of an information processing device;

FIG. 4 is a diagram showing an example of a report creation screen;

FIG. 5 is a diagram showing an example of a playback screen for endoscopic images;

FIG. 6 is a diagram showing an example of a plurality of endoscopic images acquired during examination; and

FIG. 7 is a diagram showing another example of a plurality of endoscopic images acquired during examination.

### DETAILED DESCRIPTION

The disclosure will now be described by reference to the preferred embodiments. This does not intend to limit the scope of the present disclosure, but to exemplify the disclosure.

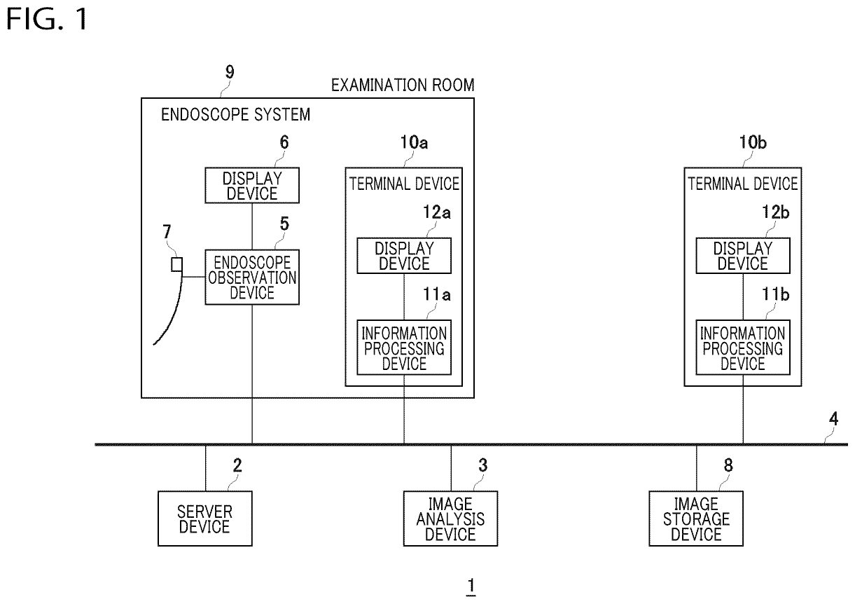

FIG. 1 shows the configuration of a medical assistance system 1 according to an embodiment. The medical assistance system 1 is provided in a medical facility such as a hospital where endoscopic examinations are performed. In the medical assistance system 1, a server device 2, an image analysis device 3, an image storage device 8, an endoscope system 9, and a terminal device 10b are communicably connected via a network 4 such as a local area network (LAN). The endoscope system 9 is installed in an examination room and has an endoscope observation device 5 and a terminal device 10a. In the medical assistance system 1, the server device 2, the image analysis device 3, and the image storage device 8 may be provided, for example, as a cloud server outside the medical facility.

The endoscope observation device 5 is connected to an endoscope 7 to be inserted into the digestive tract of a patient. The endoscope 7 has a light guide for illuminating the inside of the digestive tract by transmitting illumination light supplied from the endoscope observation device 5, and the distal end of the endoscope 7 is provided with an illumination window for emitting the illumination light transmitted by the light guide to living tissue and an imaging unit for imaging the living tissue at a predetermined cycle and outputting an imaging signal to the endoscope observation device 5. The imaging unit includes a solid-state imaging device, e.g., a CCD image sensor or a CMOS image sensor, that converts incident light into an electric signal.

The endoscope observation device 5 performs image processing on the imaging signal photoelectrically converted by a solid-state imaging device of the endoscope 7 so as to generate an endoscopic image and displays the endoscopic image on the display device 6 in real time. In addition to normal image processing such as A/D conversion and noise removal, the endoscope observation device 5 may include a function of performing special image processing for the purpose of highlighting, etc. The imaging frame rate of the endoscope 7 is preferably 30 fps or more, and may be 60 fps. The endoscope observation device 5 generates endoscopic images at a cycle of the imaging frame rate. The endoscope observation device 5 may be formed by one or more processors with dedicated hardware or may be formed by one or more processors with general-purpose hardware. The endoscope 7 according to the embodiment is a flexible endoscope and has a forceps channel for inserting an endoscopic treatment tool. By inserting biopsy forceps into the forceps channel and operating the inserted biopsy forceps, the doctor can perform a biopsy during an endoscopic examination and remove a portion of the diseased tissue.

According to the examination procedure, the doctor observes an endoscopic image displayed on the display device 6. The doctor observes the endoscopic image while moving the endoscope 7, and operates the release switch of the endoscope 7 when a biological tissue to be captured appears on the display device 6. The endoscope observation device 5 captures an endoscopic image at the time when the release switch is operated and transmits the captured endoscopic image to the image storage device 8 along with information identifying the endoscopic image (image ID). The endoscope observation device 5 may assign an image ID including a serial number to an endoscopic image in the order of capturing. The endoscope observation device 5 may transmit a plurality of captured endoscopic images all at once to the image storage device 8 after the examination is completed. The image storage device 8 records the endoscopic images transmitted from the endoscope observation device 5 in association with an examination ID for identifying the endoscopic examination.

In the embodiment, “imaging” refers to an operation of converting incident light into an electrical signal performed by a solid-state image sensor of an endoscope 7. The “imaging” may include the operation up to when the endoscope observation device 5 generates an endoscopic image from the converted electrical signal, and may further include the operation up to when the image is displayed on the display device 6. In the embodiment, “capturing” refers to an operation of acquiring an endoscopic image generated by the endoscope observation device 5. The “capturing” may include an operation of saving (recording) the acquired endoscopic image. In the embodiment, the doctor operates the release switch to thereby capture a captured endoscopic image. Alternatively, the captured endoscopic image may be automatically captured regardless of the operation of the release switch.

The terminal device 10a is installed in the examination room with an information processing device 11a and a display device 12a. The terminal device 10a may be used by doctors, nurses, and others in order to check information on a biological tissue being captured in real time during endoscopic examinations.

The terminal device 10b is installed in a room other than the examination room with an information processing device 11b and a display device 12b. The terminal device 10b is used when a doctor creates a report of an endoscopic examination. The terminal devices 10a and 10b may be formed by one or more processors having general-purpose hardware in the medical facility.

In the medical assistance system 1 according to the embodiment, the endoscope observation device 5 displays endoscopic images in real time through the display device 6, and provides the endoscopic images along with meta information of the images to the image analysis device 3 in real time. The meta information may be information that includes at least the frame number and imaged time information of each image, where the frame number indicates the number of the frame after the endoscope 7 starts imaging.

The image analysis device 3 is an electronic calculator (computer) that analyzes endoscopic images to detect lesions in the endoscopic images and performs qualitative diagnosis of the detected lesions. The image analysis device 3 may be a computer-aided diagnosis (CAD) system with an artificial intelligence (AI) diagnostic function. The image analysis device 3 may be formed by one or more processors with dedicated hardware or may be formed by one or more processors with general-purpose hardware.

The image analysis device 3 uses a trained model generated by machine learning using endoscopic images for learning, information indicating an organ and a site included in the endoscopic images, and information concerning a lesion area included in the endoscopic images as training data. Annotation work on the endoscopic images is performed by annotators with expertise, such as doctors, and machine learning may use CNN, RNN, LSTM, etc., which are types of deep learning. Upon input of an endoscopic image, this trained model outputs information indicating an imaged organ, information indicating an imaged site, and information concerning an imaged lesion (lesion information). The lesion information output by the image analysis device 3 includes at least information on the presence or absence of a lesion indicating whether the endoscopic image contains a lesion or not (shows a lesion or not). When the lesion is included, the lesion information may include information indicating the size of the lesion, information indicating the location of the outline of the lesion, information indicating the shape of the lesion, information indicating the invasion depth of the lesion, and a qualitative diagnosis result of the lesion. The qualitative diagnostic result of the lesion may include the type of lesion and includes bleeding. During an endoscopic examination, the image analysis device 3 is provided with endoscopic images from the endoscope observation device 5 in real time and outputs information indicating the organ, information indicating the site, and lesion information for each endoscopic image. Hereinafter, information indicating an organ, information indicating a site, and lesion information that are output for each endoscopic image are collectively referred to as “image analysis information.” The image analysis device 3 may generate color information (averaged color value) obtained by averaging the pixel values of the endoscopic images, and the color information may be included in the image analysis information.

When the user operates the release switch (capture operation), the endoscope observation device 5 provides the frame number, imaged time, and image ID of the captured endoscopic image to the image analysis device 3, along with information indicating that the capture operation has been performed (capture operation information). Upon acquiring the capture operation information, the image analysis device 3 provides the image ID, the frame number, the imaged time information, and image analysis information for the provided frame number to the server device 2 along with the examination ID. The image ID, the frame number, the imaged time information, and the image analysis information constitute “additional information” that expresses the features and properties of the endoscopic image. Upon acquiring the capture operation information, the image analysis device 3 transmits the additional information to the server device 2 along with the examination ID, and the server device 2 records the additional information in association with the examination ID.

When the user finishes the endoscopic examination, the user operates an examination completion button on the endoscope observation device 5. The operation information of the examination completion button is provided to the server device 2 and the image analysis device 3, and the server device 2 and the image analysis device 3 recognize the completion of the endoscopic examination.

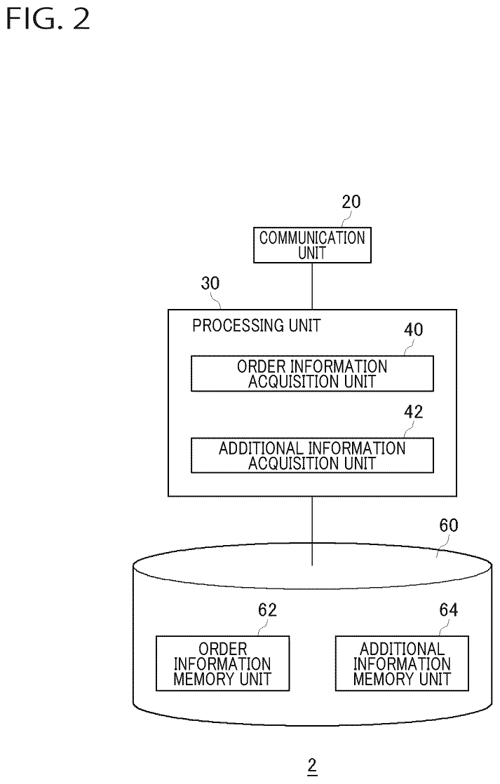

FIG. 2 shows functional blocks of the server device 2. The server device 2 includes a communication unit 20, a processing unit 30, and a memory device 60. The communication unit 20 transmits and receives information such as data and instructions between the image analysis device 3, the endoscope observation device 5, the image storage device 8, the terminal device 10a, and the terminal device 10b through the network 4. The processing unit 30 has an order information acquisition unit 40 and an additional information acquisition unit 42. The memory device 60 has an order information memory unit 62 and an additional information memory unit 64.

The server device 2 includes a computer. Various functions shown in FIG. 2 are realized by the computer executing a program. The computer includes a memory for loading programs, one or more processors that execute loaded programs, auxiliary storage, and other LSIs as hardware. The processor may be formed with a plurality of electronic circuits including a semiconductor integrated circuit and an LSI, and the plurality of electronic circuits may be mounted on one chip or on a plurality of chips. The functional blocks shown in FIG. 2 are realized by cooperation between hardware and software. Therefore, a person skilled in the art should appreciate that there are many ways of accomplishing these functional blocks in various forms in accordance with the components of hardware only, software only, or the combination of both.

The order information acquisition unit 40 acquires order information for an endoscopic examination from a hospital information system. For example, before the start of the examination work for one day at the medical facility, the order information acquisition unit 40 acquires the order information for the day from the hospital information system and stores the order information in the order information memory unit 62. Before the start of the examination, the endoscope observation device 5 or the information processing device 11a may read out order information for the examination to be performed from the order information memory unit 62 and display the order information on the display device.

The additional information acquisition unit 42 acquires the examination ID and additional information for the endoscopic image from the image analysis device 3, and stores the additional information in association with the examination ID in the additional information memory unit 64. The additional information for the endoscopic image includes an image ID, a frame number, imaged time information, and image analysis information.

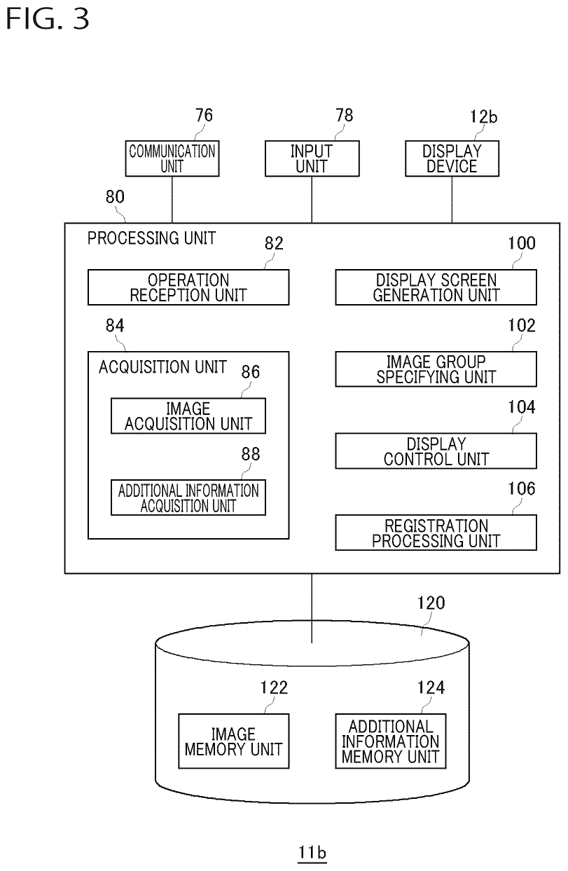

FIG. 3 shows the functional blocks of the information processing device 11b. The information processing device 11b has the function of assisting examination report creation work and includes a communication unit 76, an input unit 78, a processing unit 80, and a memory device 120. The communication unit 76 transmits and receives information such as data and instructions between the server device 2, the image analysis device 3, the endoscope observation device 5, the image storage device 8, and the terminal device 10a through the network 4. The processing unit 80 includes an operation reception unit 82, an acquisition unit 84, a display screen generation unit 100, an image group specifying unit 102, a display control unit 104, and a registration processing unit 106, and the acquisition unit 84 has an image acquisition unit 86 and an additional information acquisition unit 88. The memory device 120 has an image memory unit 122 and an additional information memory unit 124.

The information processing device 11b includes a computer. Various functions shown in FIG. 3 are realized by the computer executing a program. The computer includes a memory for loading programs, one or more processors that execute loaded programs, auxiliary storage, and other LSIs as hardware. The processor may be formed with a plurality of electronic circuits including a semiconductor integrated circuit and an LSI, and the plurality of electronic circuits may be mounted on one chip or on a plurality of chips. The functional blocks shown in FIG. 3 are realized by cooperation between hardware and software. Therefore, a person skilled in the art should appreciate that there are many ways of accomplishing these functional blocks in various forms in accordance with the components of hardware only, software only, or the combination of both.

After the completion of an endoscopic examination, the user, a doctor, inputs a user ID and a password to the information processing device 11b so as to log in. An application for creating an examination report is activated when the user logs in, and a list of already performed examinations is displayed on the display device 12b. The list of already performed examinations displays examination information such as a patient name, a patient ID, examination date and time, an examination item, and the like in a list, and the user operates the input unit 78 such as a mouse or a keyboard so as to select an examination for which a report is to be created. When the operation reception unit 82 receives an examination selection operation, the image acquisition unit 86 acquires a plurality of endoscopic images linked to the examination ID of the examination selected by the user from the image storage device 8 and stores the endoscopic images in the image memory unit 122, and the additional information acquisition unit 88 acquires additional information linked to the examination ID of the examination selected by the user from the server device 2 and stores the additional information in the additional information memory unit 124. The display screen generation unit 100 generates a report creation screen and displays the report creation screen on the display device 12b.

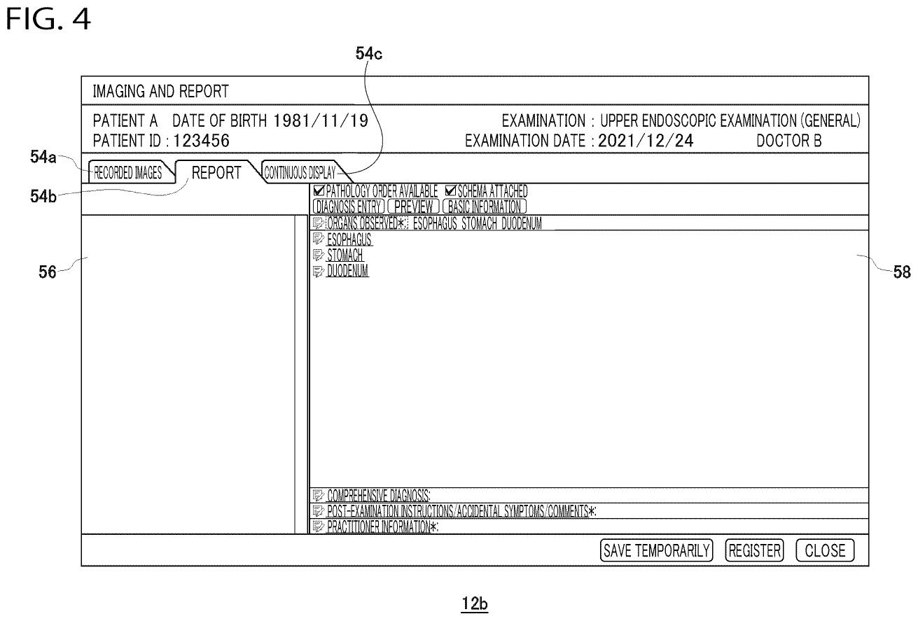

FIG. 4 shows an example of a report creation screen for inputting examination results. The report creation screen is displayed on the display device 12b while a report tab 54b is being selected. In the upper part of the screen, information such as a patient name, a patient ID, the date of birth, examination item, an examination date, and a performing doctor is displayed. These pieces of information are included in the examination order information and may be acquired from the server device 2.

The report creation screen includes two regions: an attached image display region 56 for displaying endoscopic images to be attached in a region on the left side; and an input region 58 for the user to input the examination results in a region on the right side. In the input region 58, an area is provided for entering diagnosis details for “esophagus,” “stomach,” and “duodenum,” which are observation ranges in an upper endoscopic examination. The input region 58 may have a format where a plurality of selections are displayed for examination results such that the user enters a diagnosis detail by selecting a check box or may have a free format for free text entry.

The attached image display region 56 is a region for displaying endoscopic images to be attached to a report side by side. The user selects an endoscopic image to be attached to the report from a list screen or playback screen for endoscopic images. When the user selects a recorded image tab 54a, the display screen generation unit 100 generates a list screen in which a plurality of endoscopic images captured during examination are arranged and displays the list screen on the display device 12b. When the user selects a continuous display tab 54c, the display screen generation unit 100 generates a playback screen for continuously displaying a plurality of endoscopic images acquired during examination in the order of imaging, and displays the playback screen on the display device 12b.

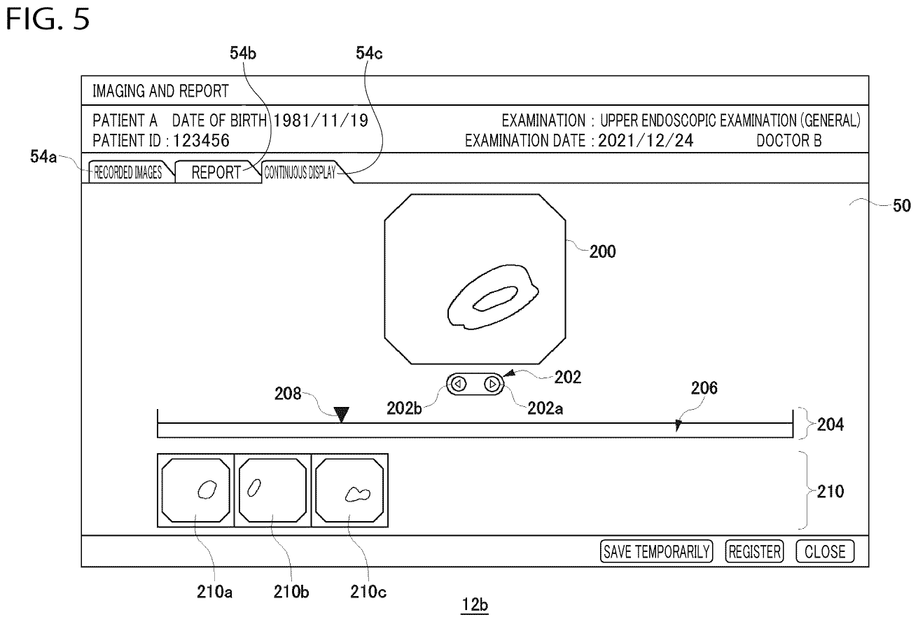

FIG. 5 shows an example of a playback screen 50 for endoscopic images. A playback region 200 for switching a plurality of endoscopic images so as to continuously display the endoscopic images is provided at the upper center part of the playback screen. A playback button 202a and a reverse playback button 202b are displayed in a playback button display region 202. When the playback button 202a is selected, endoscopic images are continuously displayed in the forward direction (the direction moving from an image with an old imaged-time toward a new image) in the playback region 200. When the reverse playback button 202b is selected, the endoscopic images are continuously displayed in the backward direction (the direction moving from an image with a new imaged time toward an old image) in the playback region 200.

The display control unit 104 displays a plurality of endoscopic images in the playback region 200 in sequence while switching between the endoscopic images when the playback button 202a or the reverse playback button 202b is selected. A pause button is displayed instead at the location of the selected playback button 202a or the reverse playback button 202b at this time. When the user operates the pause button during the continuous display of the endoscopic images, the display control unit 104 suspends the continuous display of the endoscopic images and displays a still image of the endoscope image displayed at the time of the pause button operation.

When the user places the mouse pointer on an image displayed in the playback region 200 and double-clicks the left button of the mouse, the image is selected as an attachment image and displayed in an attachment image display region 210. This example shows three attachment images 210a to 210c are being selected.

Below the playback region 200, the display screen generation unit 100 displays a horizontally-long bar display region 204 with one end indicating the imaging start time and the other end indicating the imaging end time. The bar display region 204 according to the exemplary embodiment expresses a time axis with the left end indicating the imaging start time and the right end indicating the imaging end time. The bar display region 204 may be assigned an image with the oldest imaged time to the left end and an image with the most recent imaged time to the right end so as to express the imaging order of the images. A slider 208 indicates the temporal position of an endoscopic image displayed in the playback region 200. When the user places the mouse pointer on an arbitrary position of the bar display region 204 and clicks the left button of the mouse, an endoscopic image at that time position is displayed in the playback region 200. Even when the user drags the slider 208 and drops the slider 208 at an arbitrary position in the bar display region 204, an endoscopic image at that time position is displayed in the playback region 200.

The display control unit 104 displays a band-shaped color bar 206 indicating a temporal change in color information of an imaged endoscope image in the bar display region 204. The color bar 206 in this case is configured by arranging the color information of a plurality of endoscopic images acquired during the examination in a time series manner.

In the medical assistance system 1 according to the embodiment, a situation is assumed in which a large number of endoscopic images are captured during examination. For example, when the endoscope 7 is equipped with a continuous capture (continuous shooting) function, the number of images acquired in the examination is large since the image acquisition is performed continuously while the doctor is pressing the release switch. For example, if hundreds of images are captured in a single examination, the time and effort required for the doctor to observe the images when creating an examination report are significantly increased. According to the technology disclosed in JP 2006-280792, although the observation time of a continuous image group having a high degree of similarity can be shortened, the observation time of images not included in the continuous image group cannot be shortened. Further, when images in the continuous image group include a lesion, the remaining images including a lesion other than a representative image is displayed at a high-speed display frame rate, which is not preferable. Therefore, the medical assistance system 1 according to the embodiment provides a technology for efficiently displaying images acquired during examination in order to reduce the burden of image observation performed by a doctor.

The image group specifying unit 102 has a function of specifying one or more image groups including at least one image in which a lesion is shown (hereinafter also referred to as “lesion image”) from a plurality of endoscopic images acquired during examination. The image groups may include a plurality of lesion images. The image group specifying unit 102 specifies a lesion image with reference to the additional information stored in the additional information memory unit 124, and specifies a plurality of temporally continuous images including at least two lesion images as one image group.

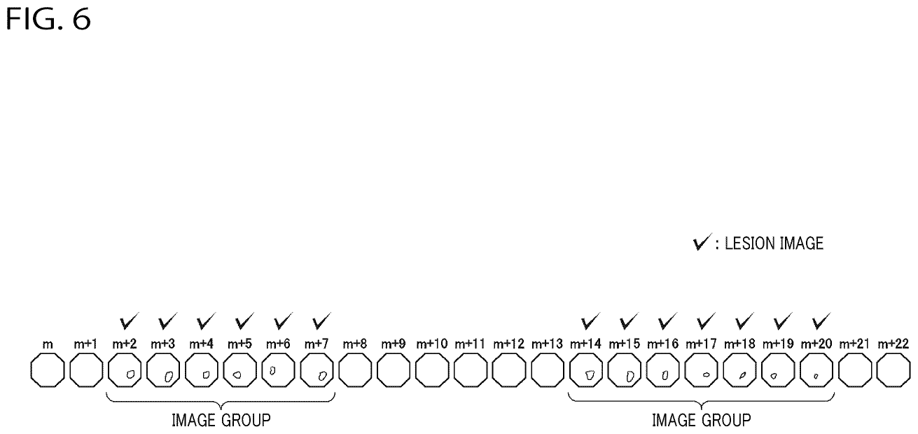

FIG. 6 shows an example in which some of a plurality of endoscopic images acquired during examination are extracted. Octagons schematically show endoscopic images, and are arranged from left in order starting from the oldest imaged time. In this example, the imaged time of an image (m) is the oldest, and the imaged time of an image (m+22) is the latest. Check marks displayed on some images indicate that a lesion is included (a lesion is shown). In the example shown in FIG. 6, images (m+2), (m+3), (m+4), (m+5), (m+6), (m+7), (m+14), (m+15), (m+16), (m+17), (m+18), (m+19), and (m+20) are lesion images. Images other than these do not show lesions.

When a plurality of lesion images are temporally contiguous, the image group specifying unit 102 specifies the continuous lesion images as one image group. In this example, the image group specifying unit 102 specifies six temporally continuous images from the image (m+2) to the image (m+7) as one image group, and specifies seven temporally continuous images from the image (m+14) to the image (m+20) as one image group.

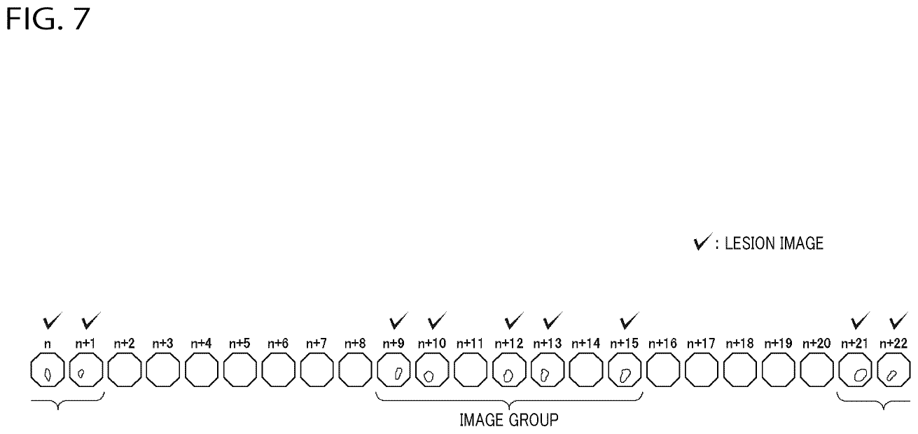

The image group specifying unit 102 may specify a plurality of temporally continuous images including at least two lesion images as one image group based on another condition. FIG. 7 shows another example in which some of a plurality of endoscopic images acquired during examination are extracted. Octagons schematically show endoscopic images, and are arranged from left in order starting from the oldest imaged time. In this example, the imaged time of an image (n) is the oldest, and the imaged time of an image (n+22) is the latest. Check marks displayed on some images indicate that a lesion is included (a lesion is shown). In the example shown in FIG. 7, images (n), (n+1), (n+9), (n+10), (n+12), (n+13), (n+15), (n+21), and (n+22) are lesion images. Images other than these do not show lesions.

The image group specifying unit 102 may specify an image group including a plurality of lesion images based on the distance between the respective imaged positions of the two lesion images. The imaged position of a lesion image may be the distal end position of the endoscope 7 at the time of the imaging of the lesion image, or may be the position of the lesion. The imaged position of the lesion image may be specified based on site information included in the image analysis information, or may be specified according to another conventional technology. The image group specifying unit 102 does not include the two lesion images in one image group if the distance between the imaged positions of the two lesion images exceeds a predetermined threshold value Dth. On the other hand, the image group specifying unit 102 includes the two lesion images in one image group if the distance between the imaged positions of the two lesion images is within the predetermined threshold value Dth.

In the example shown in FIG. 7, the image group specifying unit 102 checks the distance between the imaged position of the image (n+1) and the imaged position of the image (n+9), which is the next lesion image after the image (n+1), and determines that the image (n+1) and the image (n+9) cannot be grouped into one image group since the distance between the two imaged positions exceeds Dth.

Subsequently, the image group specifying unit 102 checks the distance between the imaged position of the image (n+9) and the imaged position of the image (n+10), which is the next lesion image after the image (n+9), and determines that the image (n+9) and the image (n+10) can be grouped into one image group since the distance between the two imaged positions is within Dth. Next, the image group specifying unit 102 checks the distance between the imaged position of the image (n+9) and the imaged position of the image (n+12), which is the next lesion image after the image (n+10), and determines that the image (n+9) and the image (n+12) can be grouped into one image group since the distance between the two imaged positions is within Dth. In the same way, the image group specifying unit 102 also checks the distance between the imaged position of the image (n+9) and the respective imaged positions of the image (n+13) and the image (n+15), and determines that the image (n+9), the image (n+13), and the image (n+15) can be grouped into one image group since the distance between the two imaged positions is within Dth in either case.

Further, the image group specifying unit 102 determines that the image (n+9) and the image (n+21) cannot be grouped into one image group since the distance between the two imaged positions exceeds Dth when checking the distance between the imaged position of the image (n+9) and the imaged position of the image (n+21). Based on the above determination result, the image group specifying unit 102 specifies the seven temporally continuous images from the image (n+9) to the image (n+15) as one image group. As described, the image group specifying unit 102 may specify an image group including a plurality of lesion images based on the distance between the respective imaged positions of two lesion images.

The image group specifying unit 102 may specify an image group including a plurality of lesion images based on the interval between the respective imaged times of the two lesion images. The image group specifying unit 102 specifies the imaged time of a lesion image with reference to the additional information stored in the additional information memory unit 124, and specifies a plurality of temporally continuous images including at least two lesion images as one image group based on the interval between the imaged times. The image group specifying unit 102 does not include the two lesion images in one image group if the interval between the imaged times of the two lesion images exceeds a predetermined threshold value Tth. On the other hand, the image group specifying unit 102 includes the two lesion images in one image group if the interval between the imaged times of the two lesion images is within the predetermined threshold value Tth.

In the example shown in FIG. 7, the image group specifying unit 102 checks the interval between the imaged time of the image (n+1) and the imaged time of the image (n+9), which is the next lesion image after the image (n+1), and determines that the image (n+1) and the image (n+9) cannot be grouped into one image group since the interval between the two imaged times exceeds Tth.

Subsequently, the image group specifying unit 102 checks the interval between the imaged time of the image (n+9) and the imaged time of the image (n+10), which is the next lesion image after the image (n+9), and determines that the image (n+9) and the image (n+10) can be grouped into one image group since the interval between the two imaged times is within Tth. Next, the image group specifying unit 102 checks the interval between the imaged time of the image (n+9) and the imaged time of the image (n+12), which is the next lesion image after the image (n+10), and determines that the image (n+9) and the image (n+12) can be grouped into one image group since the interval between the two imaged times is within Tth. In the same way, the image group specifying unit 102 also checks the interval between the imaged time of the image (n+9) and the respective imaged times of the image (n+13) and the image (n+15), and determines that the image (n+9), the image (n+13), and the image (n+15) can be grouped into one image group since the interval between the two imaged times is within Tth in either case.

Further, the image group specifying unit 102 determines that the image (n+9) and the image (n+21) cannot be grouped into one image group since the interval between the two imaged times exceeds Tth when checking the interval between the imaged time of the image (n+9) and the imaged time of the image (n+21). Based on the above determination result, the image group specifying unit 102 specifies the seven temporally continuous images from the image (n+9) to the image (n+15) as one image group. As described, the image group specifying unit 102 may specify an image group including a plurality of lesion images based on the interval between the respective imaged times of two lesion images.

The image group specifying unit 102 may also specify an image group including a plurality of lesion images based on the number of other images taken between the imaging of two lesion images. If the number of images (images that are not lesion images) included between the two lesion images exceeds a predetermined threshold value Nth, the image group specifying unit 102 does not include the two lesion images in one image group. On the other hand, if the number of images (images that are not lesion images) included between the two lesion images is within the predetermined threshold value Nth, the image group specifying unit 102 includes the two lesion images in one image group.

For example, when the threshold value Nth is set to four, since seven images are included between the image (n+1) and the image (n+9), the image group specifying unit 102 determines that the image (n+1) and the image (n+9) cannot be grouped into one image group. Further, since five images are included between the image (n+15) and the image (n+21), the image group specifying unit 102 determines that the image (n+15) and the image (n+21) cannot be grouped into one image group. On the other hand, among the images (n+9), (n+10), (n+12), (n+13), and (n+15), adjacent lesion images do not include more than four images (images that are not lesion images). The image group specifying unit 102 specifies the seven temporally continuous images from the image (n+9) to the image (n+15) as one image group.

The display control unit 104 according to the embodiment controls the display speed (display frame rate) of images in the playback region 200 based on an image group specified as described above. More specifically, the display control unit 104 displays a plurality of images included in the image group at a first display frame rate, and displays a plurality of images different from the plurality of images included in the image group (i.e., a plurality of images not included in the image group) at a second display frame rate faster than the first display frame rate. That is, the display control unit 104 displays an image group including lesion images at a relatively low first display frame rate, and displays images not included in the image group at a relatively high second display frame rate. By controlling the display frame rate by the display control unit 104 in this way, the doctor can carefully observe lesion images that require attention while efficiently observing images that do not show lesions. For example, the second display frame rate may be twice or more times the first display frame rate.

As described above, the image group may include not only lesion images but also images that do not show lesions (non-lesion images). By displaying lesion images and non-lesion images included in the image group at the same first display frame rate, the continuity of display images can be maintained, and the visibility of the continuous display of the image group can be improved.

The display control unit 104 may display only some of the plurality of images not included in the image group and hide the other images. That is, the display control unit 104 may display the plurality of images not included in the image group in a thinned-out manner. When displaying the images in a thinned-out manner, the display control unit 104 may display a non-lesion image at the same first display frame rate as that for the image group so as to maintain the continuity of the display images or may display a non-lesion image at the second display frame rate.

When the display control unit 104 specifies an image to be hidden, the registration processing unit 106 may delete the image excluded from display targets from the image memory unit 122. The image excluded from the display targets may be deleted from the image storage device 8. Thereby, the utilization efficiency of the storage area of the image memory unit 122 or the image storage device 8 can be improved.

When the image group specifying unit 102 specifies the image group, the registration processing unit 106 may delete all images not included in the image group from the image memory unit 122 such that only the images included in the image group are stored in the image memory unit 122. All images not included in the image group may be deleted from the image storage device 8.

In the report creation work, the user selects an image to be attached to a report, inputs the examination results in the input region 58 on the report creation screen, and creates the report. When the user operates a registration button (see FIG. 4), the registration processing unit 106 registers the details input on the report creation screen in the server device 2, and the report creation operation is completed.

Described above is an explanation based on the embodiments of the present disclosure. The embodiments are intended to be illustrative only, and it will be obvious to those skilled in the art that various modifications to constituting elements and processes could be developed and that such modifications are also within the scope of the present disclosure. In the embodiments, the endoscope observation device 5 transmits user-captured images to the image storage device 8. However, in an exemplary variation, the image analysis device 3 may transmit user-captured images to the image storage device 8. In the embodiment, the information processing device 11b has the processing unit 80. However, in the exemplary variation, the server device 2 may have the processing unit 80.

In the embodiments, the image analysis device 3 uses a trained model so as to detect whether or not an image includes a lesion (a lesion is shown). The image analysis device 3 may determine whether or not the image includes a lesion based on a feature amount indicating at least one of saturation, hue, shape, and size of a predetermined region in the image. At this time, the image analysis device 3 may determine the presence or absence of a lesion by image analysis without using the trained model.

The image group specifying unit 102 may specify an image group including a plurality of images taken within a predetermined imaging period. Further, the image group specifying unit 102 may specify an image group including a plurality of images of a predetermined organ or site. For example, when the doctor wishes to carefully observe an image taken of a specific site, the image group specifying unit 102 may specify an image group including a plurality of images taken during a period when the specific site was imaged, and the display control unit 104 may continuously display a plurality of images of the site at the first display frame rate.

In the embodiments, a method has been described that is for efficiently displaying a plurality of images acquired by using an endoscope 7 that is inserted into the patient's gastrointestinal tract by a doctor. This method can be applied when displaying a plurality of images acquired by a capsule endoscope with an imaging frame rate greater than 2 fps. For example, if the imaging frame rate is 8 fps and when the inside of the body is imaged over about right hours, about 230,000 images of the inside of the body will be acquired. This method can be effectively applied in a capsule endoscopic examination since the number of images that are acquired is enormous.

What is claimed is:

1. A medical assistance system comprising:
a processor comprising hardware, wherein
the processor is configured to:
specify one or more image groups including at least one lesion image showing a lesion from among a plurality of images acquired during examination, the image groups being configured in time series starting from an image in which the lesion is first framed;
display the image groups at a first display frame rate; and
display images different from those in the image groups among the plurality of images at a second display frame rate higher than the first display frame rate or display the plurality of images different from those in the image groups in a thinned-out manner.

2. The medical assistance system according to claim 1, wherein
the image group ends with an image immediately before an image in which the lesion is framed out in time series.

3. The medical assistance system according to claim 1, wherein
the processor is configured to:
specify the image group including the plurality of lesion images based on at least one of the distance between respective imaged positions of two of the lesion images, the interval of imaged time between the two lesion images, and the number of other images imaged between respective imaging instances of the two lesion images.

4. The medical assistance system according to claim 1, wherein
whether or not the image shows a lesion is determined based on a feature amount indicating at least one of saturation, hue, shape, and size of a predetermined region in the images.

5. The medical assistance system according to claim 1, wherein
the processor is configured to:
specify the image group including at least one of an image taken of a predetermined organ or site or an image taken within a predetermined imaging period.

6. The medical assistance system according to claim 1, having:
a memory unit that stores the plurality of images acquired during the examination, wherein
the processor is configured to:
save only images included in the specified image groups in the memory unit.

7. The medical assistance system according to claim 1, wherein
the plurality of images acquired during the examination are images taken by a capsule endoscope with an imaging frame rate greater than 2 fps.

8. The medical assistance system according to claim 1, wherein
the plurality of images acquired during the examination are images taken by an endoscope with an imaging frame rate of 30 fps or more.

9. An image display method, comprising:
specifying one or more image groups including at least one lesion image showing a lesion from among a plurality of images acquired during examination, the image groups being configured in time series starting from an image in which the lesion is first framed;
displaying the image groups at a first display frame rate; and
displaying images different from those in the image groups among the plurality of images at a second display frame rate higher than the first display frame rate or displaying the plurality of images different from those in the image groups in a thinned-out manner.

10. A recording medium having recorded thereon a program, comprising computer-implemented modules including:
a module that specifies one or more image groups including at least one lesion image showing a lesion from among a plurality of images acquired during examination, the image groups being configured in time series starting from an image in which the lesion is first framed;
a module that display the image groups at a first display frame rate; and
a module that displays images different from those in the image groups among the plurality of images at a second display frame rate higher than the first display frame rate or displays the plurality of images different from those in the image groups in a thinned-out manner.
